(19) United States
(12) Patent Application Publication
(10) Pub. No.: US 2026/0108217 A1
(43) Pub. Date: Apr. 23, 2026

(54) SYSTEM AND METHODS FOR SYNCHRONIZATION OF BIOLOGICAL DATA IN REAL TIME WITH MILLISECOND TEMPORAL PRECISION

(71) Applicant: HUN-REN KÍSÉRLETI ORVOSTUDOMÁNYI KUTATÓINTÉZET, Budapest (HU)

(72) Inventors: Balázs György HANGYA, Budapest (HU); Tamás LASZLOVSZKY, Pomáz (HU); András SZÉLL, Gárdony (HU)

(21) Appl. No.: 19/109,300

(22) PCT Filed: Sep. 5, 2023
(86) PCT No.: PCT/HU2023/050054

§ 371 Date: Mar. 6, 2025

(30) Foreign Application Priority Data
Sep. 6, 2022 (HU) P2200356

(51) Int. Cl. A61B 5/00; A61B 5/291; A61B 5/296; A61B 5/378; A61B 5/395

(52) U.S. Cl. CPC A61B 5/7285; A61B 5/291; A61B 5/296; A61B 5/378; A61B 5/395

(57) ABSTRACT

The present invention relates to a system (1) for synchronization of biological data in real time with millisecond temporal precision. The system comprises a screen (10) for displaying visual stimuli; a control unit (20) connected to the screen (10) for transmitting visual stimuli; an optical sensor (30) connected to the screen (10) for detecting visual stimuli; a neuronal activity measuring device (40) with an internal clock (41), which is connected to the control unit (20) for transmitting neuronal activity registered by the internal clock (41); feedback device (50), which comprises a plurality of push buttons (51) suitable for detecting responses of a subject to visual stimuli, and a stop button (52) suitable for stopping the display of visual stimuli. The system (1) is characterized by the fact that it comprises a microcontroller (60) with a real-time clock (61), which is connected to the optical sensor (30) and the feedback device (50) for registering their events in real time, with millisecond precision, using the real-time clock (61), wherein the microcontroller (60) is capable of generating a synchronizing time signal for each registered event, and the microcontroller (60) is connected to the control unit (20) for transmitting the registered events and their associated synchronizing time signals; wherein the control unit (20) is configured such that it is suitable for receiving each event registered by the real-time clock (61) and the neuronal activity events registered by the internal clock (41), as well as for their correlation based on the synchronizing time signals. The system further comprises optoisolator (70) suitable for providing the electrical isolation of the connection between the microcontroller (60) and the neuronal activity measuring device (40).

The present invention also relates to methods for a system (1) for synchronization of biological data of a subject in real time with millisecond temporal precision.

This invention relates to a system and methods for synchronization of biological data in real time with millisecond temporal precision.

In order to establish the status of certain neurological diseases, such as dementia, Parkinson's disease, Alzheimer's disease, it is necessary to determine both motor symptoms and cognitive deviations. Among other things, the treatment guidelines used in the case of the above-mentioned neurological diseases are primarily aimed at treatment based on the assessment of motor functions. However, the treatment of cognitive functions is relegated to the background, as it can generally be stated that the assessment of the cognitive state is limited to filling out clinical questionnaires, which may be suitable for establishing a general mental status but cannot replace the conclusions from the results of quantitative psychological and neurological measurements. One of the state-of-the-art methods for determining the psychophysical state of mammals, especially humans, is the registration and analysis of certain stimuli/series of stimuli, the responses to the stimuli and the neuronal activity measured during the stimulation, i.e., the spike pattern (action potentials) of neurons in real time, with millisecond temporal precision. Real-time, millisecond precision measurement at the hardware level cannot be realized with standard devices suitable for providing sensory stimuli and measuring behavioral and neuronal responses due to the time difference arising from the operation and data transmission of the individual devices.

The Psyhchtoolbox-3 software package (link: https://www.psychtoolbox.net/) is a Matlab-based solution that can be used to perform behavioral experiments and measure the response time and performance of subjects. The solution is suitable for determining the visual and auditory stimuli used in laboratory experiments, as well as the responses to them, with millisecond time precision, which is implemented at the software level. The advantage of the solution is that it realizes the real-time management of different types of stimuli and responses, however, the temporal precision is highly dependent on the current software and hardware environment, which makes it necessary to adjust the Matlab-based algorithm to the experimental parameters for efficient operation.

U.S. Pat. No. 7,509,253 relates to a device for measuring latency between an audible stimulus and a human subject's response (speech). The device according to the invention logs the time between the end of the stimulus and the beginning of the subject's speech reaction on its clock and then uses a computing unit to calculate the time elapsed until the response. Although the device determines the delay between stimulus and response at the hardware level, the device is configured to test the subject's reaction time and is not capable of handling data from individual devices in real time.

U.S. 2012010474 relates to a method, system and computer program for analyzing the response of a subject to an input stimulus with a temporal resolution below 160 milliseconds. The method comprises the steps of automatically determining the test person's response to the input stimulus presented on the display, and synchronising said response with the input stimulus, wherein the response is essentially brain activity data. The invention is characterized by that the responses to the calibration stimuli are also taken into account during the data analysis to synchronize the stimulus and response. During the calibration step, an adaptive algorithm is applied to create a database based on the response of several subjects to the input stimulus, wherein, based on the stored data, the adaptive algorithm automatically determines the response of an additional subject or its characteristic and matches it with the input stimulus in time during synchronization. The solution according to the invention synchronizes the brain activity and the input stimuli, but not in real time, in a subject-specific manner, but based on matching the responses of previous subjects to the stimuli.

None of the above solutions enables the synchronization of stimuli from different devices, responses to stimuli, and brain activity at a hardware level, in real time, with millisecond temporal precision.

The object of the solution according to the invention is to provide a system and methods that enable real time synchronization of biological data with millisecond temporal precision, more specifically data from human psychophysical tests, i.e., visual stimuli, the subject's responses to the visual stimuli and the neuronal activity measured during stimulation with a real-time clock, and the determination of the subject's psychophysical status based on the obtained and/or synchronized data.

The object of the present invention is to provide a system for synchronization of biological data in real time, with millisecond time precision,

said system comprises:
a screen for displaying visual stimuli;
a control unit connected to the screen for transmitting visual stimuli;
an optical sensor connected to the screen for detecting visual stimuli;
a neuronal activity measuring device with an internal clock, which is connected to the control unit for transmitting neuronal activity registered by the internal clock;
feedback device, which comprises:
a plurality of push buttons suitable for detecting responses of a subject to visual stimuli, and
a stop button suitable for stopping the display of visual stimuli;
characterized in that the system comprises
a microcontroller with a real-time clock, which
is connected to the optical sensor and the feedback device for registering their events in real time, with millisecond precision, using the real-time clock, wherein the microcontroller is capable of generating a synchronizing time signal for each registered event, and
is connected to the control unit for transmitting the registered events and their associated synchronizing time signals;
wherein the control unit is configured such that it is suitable for receiving each event registered by the real-time clock and the neuronal activity events registered by the internal clock, as well as for their correlation based on the synchronizing time signals,
optoisolator suitable for providing the electrical isolation of the connection between the microcontroller and the neuronal activity measuring device.

The neuronal activity measuring device can be an EEG, EMG, intraoperative extracellular electrophysiological measuring device and/or any other device suitable for recording neuronal activity, which has an internal clock for temporal registration of neuronal activity.

Start times and end times of the display of the visual stimuli can be detected by the optical sensor.

Preferably, the microcontroller having the real-time clock is suitable for registering at least the following events in real time, with millisecond precision, and producing the associated synchronizing time signal: the start times of the display of the visual stimuli measured by the optical sensor and push button signals for the start times of visual perception of a subject.

Preferably, the microcontroller having the real-time clock is also suitable for registering the following events in real time, with millisecond precision, and producing the associated synchronizing time signal: the end times of the display of the visual stimuli measured by the optical sensor and push button signals for the end times of visual perception of a subject, optionally time of the stop button signal.

Preferably, the neuronal activity measuring device having the internal clock is suitable for registering the following events: neuronal activity at the start times of the display of each visual stimulus, neuronal activity at the end times of the display of each visual stimulus, and action potentials measured during the display of each visual stimulus, especially start times, end times and/or peak times of the action potentials.

The control unit can be connected to the microcontroller via wired connection, preferably via serial port.

Preferably, the connection between the microcontroller and the neuronal activity measuring device is a wired connection, preferably a trigger cable.

The control unit may be suitable for receiving and correlating the registered events, the registered neuronal activity and the synchronizing time signals; storing, combining and processing received and/or correlated events and neuronal activity.

Additionally, it is an object of the present invention to provide a method for a system for synchronization of biological data of a subject in real time, with millisecond time precision, said method comprising:
providing a control unit for controlling visual stimuli,
providing a screen and transmitting the visual stimuli to the screen for display,
providing an optical sensor, wherein using the optical sensor to detect the display of visual stimuli on the screen,
providing a feedback device, which comprises a plurality of push buttons and a stop button, wherein the subject gives feedback on the perception of the displayed visual stimuli by pressing one of the push buttons,
providing a neuronal activity measuring device with an internal clock, thereby registering the neuronal activity of said subject during the display of visual stimuli,
providing a microcontroller, which comprises a real-time clock, thereby registering events of the optical sensor and the feedback device in real time, with millisecond precision, and generating a synchronizing time signal for each of them,
transmitting the events and neuronal activity registered by the internal clock and the real-time clock, as well as the synchronizing time signals to the control unit,
using the synchronizing time signals to correlate at least the events registered by the real-time clock, preferably also the neuronal activity events registered by the internal clock.

Preferably, the method comprising:
using the real-time clock of the microcontroller to register, in real time, with millisecond precision, start times of the display of the visual stimuli measured by the optical sensor and push button signals for start times of visual perception of the subject,
generating a first synchronizing time signal for the registered start times of the display of each visual stimulus measured by the optical sensor, and a second synchronizing time signal for each registered push button signal, which are related to the start times of the subject's visual perception,
transmitting the registered start time events and the first and second synchronizing time signals to the control unit,
based on the first and second synchronizing time signals, correlating the registered start time events by the control unit.

Preferably, the method comprising:
using the real-time clock of the microcontroller to register, in real time, with millisecond precision, end times of the display of visual stimuli measured by the optical sensor and push button signals for end times of visual perception of the subject,
generating a third synchronizing time signal for the registered end times of the display of each visual stimulus measured by the optical sensor, and a fourth synchronizing time signal for the registered push button signals, which are related to the end times of the subject's visual perception,
transmitting the registered end time events and the third and fourth synchronization time signals to the control unit,
based on the third and fourth synchronizing time signals, correlating the registered end time events by the control unit.

Preferably, the method comprising:
using the real-time clock of the microcontroller to register, in real time, with millisecond precision, time of pressing the stop button,
generating a fifth synchronizing time signal for registered time of pressing the stop button,
transmitting the registered stop button event and the fifth synchronization time signal to the control unit,
based on the third, fourth and fifth synchronizing time signals, correlating the registered end time events and the registered stop button event by the control unit.

Preferably, the method comprising:
using the internal clock of the neuronal activity measuring device to register the neuronal activity during each registered start time event, the neuronal activity during each registered end time events, and particularly start times, end times and/or peak times of the action potentials measured during the display of each visual stimulus,
transmitting the neuronal activity registered by the neuronal activity measuring device to the control unit,
based on the first, second, third, fourth and/or fifth synchronizing time signal, by the control unit, correlating the neuronal activity registered by the internal clock of the neuronal activity measuring device and at least one of the events registered by the real-time clock of the microcontroller.

Preferably, the method comprising:
by the control unit, combining the correlated events and neuronal activity, preferably combining the events and neuronal activity that are correlated based on the first, second, third, fourth and fifth synchronizing time signals.

Preferably, the method comprising:
by the control unit, processing at least one, preferably all, of the registered, correlated and/or combined events and neuronal activity to determine quantitative characteristics of the subject's psychophysical status.

Preferably, the method comprising:
by the control unit, comparing the quantitative characteristics of the subject's psychophysical status with predetermined quantitative characteristics and/or quantitative characteristics of psychophysical statuses stored in a database, wherein the database comprises quantitative characteristics of the psychophysical status of standard subjects and/or subjects suffering from a given disease, and
based on the result of the comparison, clustering the quantitative characteristics in order to determine the subject's psychophysical status.

Furthermore, the aim of the invention has been achieved by implementing a method for a system synchronization of biological data of a subject in real time, with millisecond time precision, said method comprising:
using real-time clock of the microcontroller to register in real time, with millisecond precision, start times of the display of the visual stimuli measured by the optical sensor and push button signals for start times of the visual perception of the subject,
generating a first synchronizing time signal for the registered start times of the display of each visual stimulus measured by the optical sensor, and a second synchronizing time signal for each registered push button signal, which are related to the start times of the subject's visual perception,
transmitting the registered start time events and the first and second synchronizing time signals to the control unit,
based on the first and second synchronizing time signals, correlating the registered start time events by the control unit.

Preferably, the method comprising:
using the real-time clock of the microcontroller to register, in real time, with millisecond precision, end times of the display of visual stimuli measured by the optical sensor and push button signals for end times of the visual perception of the subject,
generating a third synchronizing time signal for the registered end times of the display of each visual stimulus measured by the optical sensor, and a fourth synchronizing time signal for the registered push button signals, which are related to the end times of the subject's visual perception,
transmitting the registered end time events and the third and fourth synchronization time signals to the control unit,
based on the third and fourth synchronizing time signals, correlating the registered end time events by the control unit.

Preferably, the method comprising:
using the real-time clock of the microcontroller to register, in real time, with millisecond precision, time of pressing the stop button,
generating a fifth synchronizing time signal for registered time of pressing the stop button,
transmitting the registered stop button event and the fifth synchronization time signal to the control unit,
based on the third, fourth and fifth synchronizing time signals, correlating the registered end time events and the registered stop button event by the control unit.

Preferably, the method comprising:
using the internal clock of the neuronal activity measuring device to register the neuronal activity during each registered start time event, the neuronal activity during each registered end time event, and particularly start times, end times and/or peak times of the action potentials measured during the display of each visual stimulus,
transmitting the neuronal activity registered by the neuronal activity measuring device to the control unit,
based on the first, second, third, fourth and/or fifth synchronizing time signal, by the control unit, correlating the neuronal activity registered by the internal clock of the neuronal activity measuring device and at least one of the events registered by the real-time clock of the microcontroller.

Preferably, the method comprising:
by the control unit, combining the correlated events and neuronal activity, preferably combining the events and neuronal activity that are correlated based on the first, second, third, fourth and fifth synchronizing time signals.

Preferably, the method comprising:
processing at least one, preferably all, of the registered, correlated and/or combined events and neuronal activity to determine quantitative characteristics of the subject's psychophysical status.

Preferably, the method comprising:
comparing the quantitative characteristics of the subject's psychophysical status with predetermined quantitative characteristics and/or quantitative characteristics of psychophysical statuses stored in a database, wherein the database comprises quantitative characteristics of the psychophysical status of standard subjects and/or subjects suffering from a given disease, and
based on the result of the comparison, clustering the quantitative characteristics in order to determine the subject's psychophysical status.

In the following, definitions that help to understand the description of the invention are described.

The microcontroller's real-time clock (RTC) is an electronic device, typically an integrated circuit, which enables real-time timestamping, i.e., real-time logging, of the events of the devices connected to it, with high precision and accuracy, preferably millisecond temporal precision, with low energy consumption.

With the real-time clock of the microcontroller, the following events can be registered: the start and end times of the display of visual stimuli measured by the optical sensor, push button signals, which refer to the start times and end times of the subject's visual perception, and optionally the time of the stop button event, i.e., the time of pressing the stop button. Related to this, the registered start time events mean the start times of the display of the visual stimuli measured by the optical sensor, and the push button signals refer to the start times of the subject's visual perception of the displayed visual stimuli. Furthermore, the registered end time events mean the end times of the display of the visual stimuli measured by the optical sensor, and the push button signals refer to the end times of the subject's visual perception of the displayed visual stimuli.

The neuronal activity measuring device is an EEG (electroencephalograph), EMG (electromyograph), intraoperative extracellular electrophysiological measuring device and/or any other device suitable for recording neuronal activity that has an internal clock for temporal registration of neuronal activity. The time registration by the device's internal clock is typically done with a time difference compared to real time.

In the context of the present invention, events are synchronized by means of synchronizing time signals generated by the microcontroller's real-time clock. A synchronizing time signal is generated for each event registered with the microcontroller's real-time clock, which essentially assigns a real-time, millisecond precision time stamp to the given registered event. On the one hand, the synchronizing time signal enables the correlation, i.e., time-based matching, of the events of the different devices with each other, which events are registered by the microcontroller, and on the other hand, the correlation with the events registered by the internal clock of the neuronal activity measuring device, so the neuronal activity events that are not registered in real time, i.e., in relative time, can also be determined in real time through the synchronizing time signals.

Combining the correlated events means that after correlating at least two of the registered start time events, registered end time events, registered stop button event, and registered neuronal activities, several correlated event-event or correlated event-neuronal activity pairs can be selected to combine with each other. The correlation of all registered events and registered neuronal activity corresponds to the combination of all correlated pairs, i.e., if the registered start time events, registered end time events, registered stop button event, and the neuronal activity registered during the above mentioned events, then all registered events and neuronal activity can be determined in real time with millisecond precision and can be matched and assigned to each other. This essentially means that in order to determine the psychophysical status of a subject, it is not automatically necessary to correlate, i.e., match, all registered events and neuronal activity, however, when combining, it is advantageous if the events registered during the presentation of the visual stimuli and the neuronal activity measured during the duration of the visual stimuli are also taken into account, so all specific behavioral and electrophysiological information is simultaneously documented and then processed as follows.

The processing of the data means the evaluation of at least one, preferably all, of the registered, correlated and/or combined events in such a way that quantitative characteristics of the psychophysical status of the tested subject are determined. During the processing, quantified quantitative characteristics can be assigned to each of the above mentioned events.

The quantitative characteristics can be compared with predetermined quantitative characteristics that are related to a psychophysical status and/or neurological deviation, or can be compared with quantitative characteristics stored in a database, which comprise the quantitative characteristics of standard, i.e., healthy subjects, and/or subjects suffering from a specific disease, typically a neurological disease. Such a predetermined characteristic or a characteristic stored in a database can be, for example, the time delay of the subject's response to the stimulus, the pattern of neuronal activity measured during the display of the stimulus, etc. Based on the results of the comparison, clustering, i.e., classification, can be performed to determine the psychophysical status of the tested subject.

Hereinafter, the preferred exemplary embodiments of the invention are described with the help of the accompanying drawing. In the drawing:

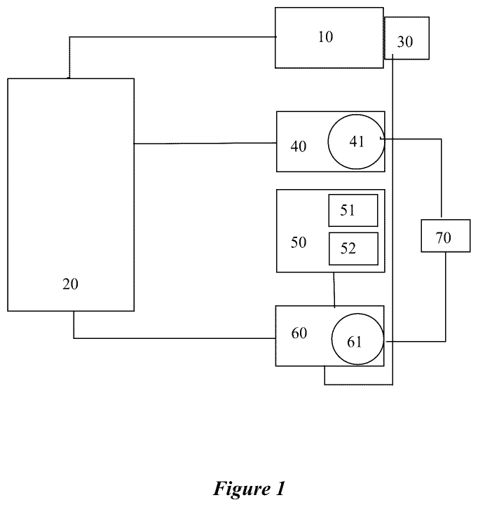

FIG. 1 schematically represents a preferred system according to the invention,

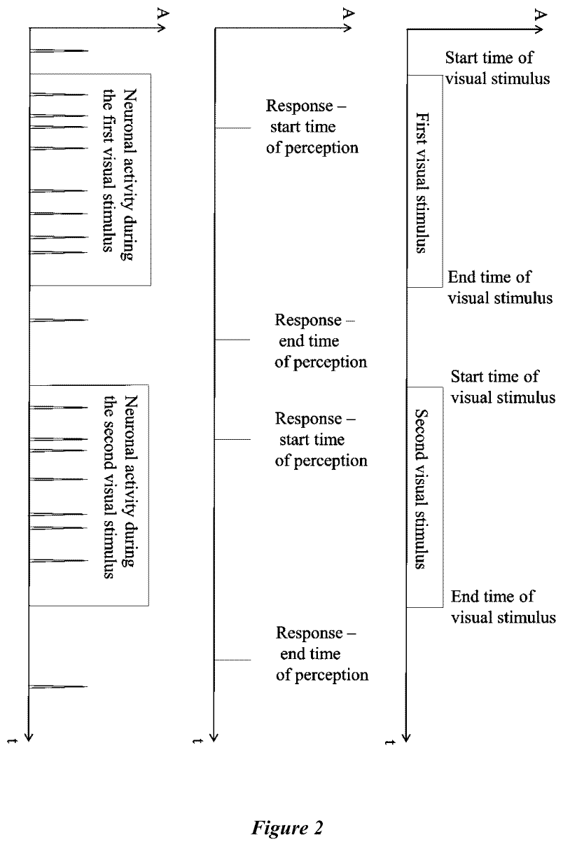

FIG. 2 schematically illustrates an example of events and neuronal activity registered by the real-time clock of the microcontroller and the internal clock of the neuronal activity measuring device, which are matched to each other,

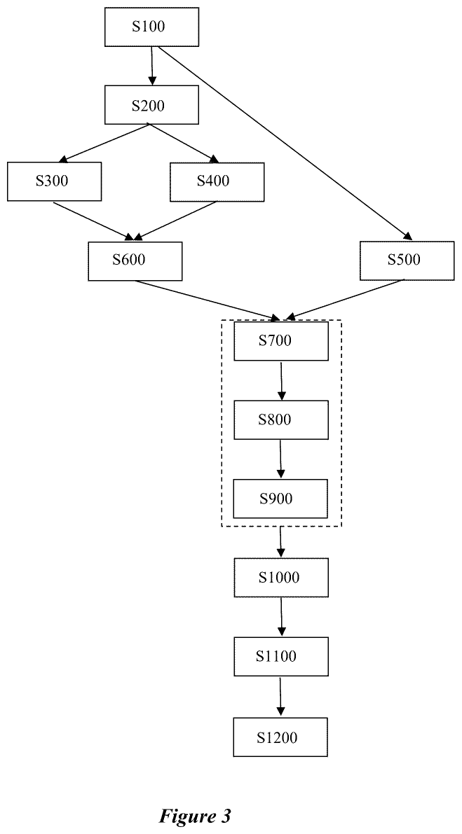

FIG. 3 shows a flow diagram of the steps of a method according to the invention, and

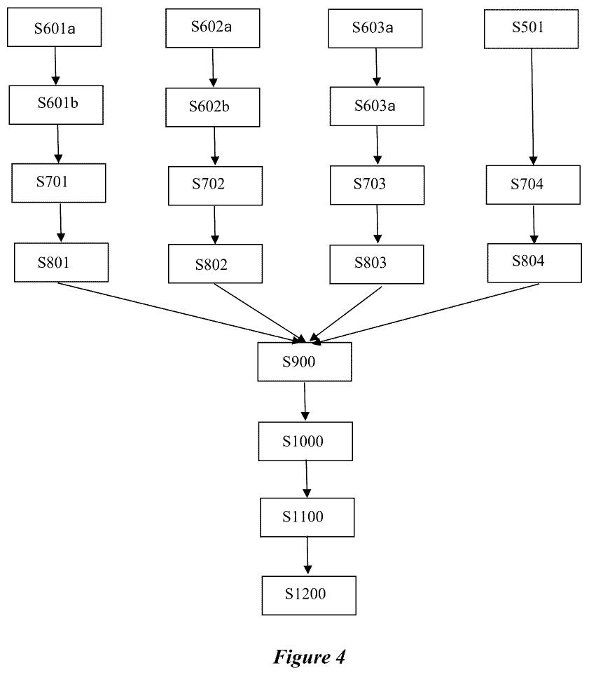

FIG. 4 also shows a flow diagram of the steps of a further method according to the invention.

FIG. 1 schematically shows the interconnection of individual elements of a preferred system 1 according to the invention. The system 1 is used for real-time synchronization of biological data with millisecond temporal precision, wherein the biological data originate from a human subject. The system 1 enables the display of visual stimuli, the registration of the subject's responses to the visual stimuli and the registration of the subject's neuronal activity during the visual stimulation, and then the real-time correlation, i.e., synchronization, of the registered data with millisecond precision. The system 1 comprises a screen 10 for displaying visual stimuli to achieve the above objective. The screen 10 can typically be a computer display.

The system 1 also comprises a control unit 20 which is connected to the screen 10 for transmitting visual stimuli. The control unit 20 preferably stores several visual stimuli, which can be, for example, images or videos. The control unit 20 is also suitable for transmitting feedback related to a subject's responses to the perception of a given visual stimulus to the screen 10, with the aim of displaying the feedback on the screen 10. The control unit 20 also performs all the control processes of the system 1, which will be described in detail later.

The system 1 comprises an optical sensor 30 connected to the screen 10 for detecting visual stimuli. The optical sensor 30 is preferably attached to one side of the screen 10, where it can detect the display of a visual stimulus, i.e., the start time of the display, and the end of the display of a visual stimulus, i.e., the end time of the display.

The system 1 comprises a neuronal activity measuring device 40 with an internal clock 41 which is connected to the control unit 20 for transmitting neuronal activity, which is registered by the internal clock 41 in a time-based manner. The neuronal activity measuring device 40 can be an EEG, EMG, intraoperative extracellular electrophysiology measuring device and/or any other device suitable for recording neuronal activity, which has an internal clock 41 for temporal registration of neuronal activity. The neuronal activity measuring device 40 with its internal clock 41 can be suitable for registering the following events: neuronal activity measured at the start time of the display of each visual stimulus, neuronal activity measured at the end time of the display of each visual stimulus, and preferably the action potentials measured during the display of each visual stimulus, especially start times, end times and/or peak times of the actional potentials. Due to the operation of the device 40 itself, the internal clock 41 of the neuronal activity measuring device 40 does not record the neuronal activity in real time, and due to the transmission of the data to the control unit 20, a time difference occurs between the actually generating neuronal activity and the recorded and then transmitted neuronal activity.

The system 1 comprises a feedback device 50, which comprises a plurality of push buttons 51 for providing the subject's response to the visual stimuli, including indicating the start and end of the visual stimulus, but can also be used to provide other types of responses related to the perception of visual stimuli. Depending on the experimental goals, a subject can use a single push button 51 of the feedback device 50 or all push buttons 51 to provide a response, for example, the subject can provide adequate feedback on the sequence and/or timing of the appearance of the visual stimuli on the screen 10 by pressing the push buttons 51 in the appropriate order. The feedback device 50 also comprises a stop button 52, with which the display of the visual stimuli on the screen 10 can be stopped manually.

The system 1 comprises a microcontroller 60 having a real-time clock 61. The microcontroller 60 is connected to the optical sensor 30 and the feedback device 50 to register the visual stimuli detected by the optical sensor 30 and the responses given by the subject with the feedback device 50, in real time, with millisecond temporal precision, using the real-time clock 61. Microcontroller 60 generates a synchronization time signal for each registered event, essentially tags these events with a real-time time stamp, thus enabling real-time determination of these events. The microcontroller 60 having the real-time clock 61 can be suitable for registering at least the following events in real time, with millisecond accuracy, and for producing a synchronizing time signal related to them: the start times of the display of the visual stimuli measured by the optical sensor 30 and push button signals, which refer to the start times of the subject's visual perception. The microcontroller 60 having the real-time clock 61 can also be suitable for registering the following events in real time, with millisecond accuracy, and for producing a synchronizing time signal related to them: the end times of the display of the visual stimuli measured by the optical sensor 30 and push button signals, which refer to the end times of the subject's visual perception, and optionally the time of the stop button signal.

The microcontroller 60 can be integrated into the feedback device 50—in FIG. 1 they are separated from each other-for a space-saving, compact design. The microcontroller 60 is connected to the control unit 20 to transmit the events registered by the real-time clock 61 and the synchronization signals associated with them. The microcontroller 60 can be wired to the control unit 20, preferably via a serial port. The control unit 20 is therefore connected to the screen 10 for transmitting visual stimuli and optionally feedbacks related to the visual stimuli, and is also connected to the neuronal activity measuring device 40 to receive the neuronal activity registered with its internal clock 41, and is connected to the microcontroller 60 to receive events registered with its real-time clock 61 and receive synchronizing time signals generated in connection with these registered events from the microcontroller 60, wherein the synchronizing time signals can be transmitted from the microcontroller 60 to the control unit 20 via wired connection, preferably via a serial port. Based on the synchronizing time signals, the control unit 20 also correlates the individual registered events with each other and with the neuronal activity, the possible correlations of the individual events are presented in detail below. The control unit 20 can also be suitable for storing, combining and processing received registered and/or correlated events and/or neuronal activities.

The system 1 comprises an optoisolator 70, which is suitable for the electrical insulation of the wired connection—which is preferably a trigger cable—between the microcontroller 60 and the neuronal activity measuring device 40, so that these two devices can operate safely at the same time during the use of the system 1. As shown in system 1 according to FIG. 1, the optoisolator 70 is arranged along the wired connection between the microcontroller 60 and the neuronal activity measuring device 40 in such a way as to interrupt the wired connection between the microcontroller 60 and the neuronal activity measuring device 40, thus ensuring electrical insulation between the two elements.

FIG. 2 schematically illustrates, with the help of diagrams, the correlation of events registered with the real-time clock 61 of the microcontroller 60 and the internal clock 41 of the neuronal activity measuring device 40, wherein the x-axis in each diagram represents time (t), while the y-axis represents amplitude (A). The upper diagram shows the start times and end times of the appearance of the visual stimuli detected by the optical sensor 30 on the screen 10 for each visual stimulus. The middle diagram shows when the visual stimuli displayed on the screen 10 are perceived by a subject, wherein the subject can give responses with the push buttons 51 of the feedback device 50 of the system 1. As shown in the upper and middle diagrams, there is a time difference between the appearance of the visual stimuli on the screen 10 and the perception by the subject, which corresponds to the subject's reaction time required for stimulus processing and response. In addition to detecting the appearance of visual stimuli with the optical sensor 30 and registering the subject's responses to the appearance of the stimuli with the push buttons 51, the system 1 is suitable for registering the disappearance of individual visual stimuli on the screen 10, i.e., the end times of the appearance of visual stimuli, as well as the time of perception of these end times by a subject. As shown in the upper and middle diagram of FIG. 2, it can also be seen that there is a time difference between the detection of the end times with the optical sensor 30 and the subject's response to these end times with the push buttons 51, which corresponds to the subject's reaction time related to the perception of each end time. According to FIG. 2, although the mentioned start times and end times were also registered, with the solution according to the invention it is not absolutely necessary to register the end time events, this may change depending on the experimental goals. The events shown in the upper and middle diagrams, i.e., the start times of the display of the visual stimuli measured by the optical sensor 30 and the push button signals, which refer to the start times of the subject's visual perception, as well as the end times of the display of the visual stimuli measured by the optical sensor 30 and the push button signals, which refer to the end times of the subject's visual perception, are registered with the real-time clock 61 of the microcontroller 60 in real time, with millisecond temporal precision. The microcontroller 60 generates a synchronizing time signal for each aforementioned event—not shown in FIG. 2—which is transmitted to the control unit 20 together with the events registered in real time.

Although not shown in FIG. 2, the pressing of the stop button 52 is also registered with the real-time clock 61. By pressing the stop button 52, the display of the visual stimulus on the screen 10 is cancelled, so in this case the time of pressing the button and the disappearance of the visual stimulus coincide.

The lower diagram shows neuronal activity, i.e., action potentials, measured by the internal clock 41 of the neuronal activity measuring device 40. The action potentials are generated before and after the appearance of the individual visual stimuli in accordance with the physiological function, however, they occur with a higher frequency during the individual visual stimuli, which reflects the electrophysiological changes in a subject during the stimulation. The neuronal activities registered with the internal clock 41 of the neuronal activity measuring device 40 are also transmitted to the control unit 20, wherein, based on the synchronization time signals received from the microcontroller 60, the control unit 20 correlates the individual registered events with each other and with the neuronal activity, i.e., matches them with each other in real time. The lower diagram of FIG. 2 shows the window indicating the duration of each visual stimulus that the control unit 20 can determine on the basis of the synchronizing time signals in order to determine, in real time, the start and end time events detected by the optical sensor 30, the start and end time events indicated by push buttons 51, and the action potentials measured by the 40 neuronal activity measuring devices, and then match them with each other, i.e., correlate them.

FIG. 2 shows two visual stimuli and their corresponding responses given by the subject, as well as the neuronal activity measured during them, but both the system and the methods according to the invention are suitable for handling more than two visual stimuli and multiple responses in connection with them, as well as neuronal activity measured during them.

FIG. 3 shows a flow diagram of the steps of a method according to the invention. The method according to FIG. 3 refers to the application of a system 1 presented above, which is used to synchronize the biological data of a subject in real time, with millisecond precision.

Said method comprises the following steps: in a step S100, providing a control unit 20 for controlling visual stimuli, preferably for storing and transmitting them.

In step S200, providing a screen 10 and transmitting the visual stimuli to the screen in order to display them.

In step S300, providing an optical sensor 30, which is attached to the screen 10, wherein the optical sensor 30 is used to detect the display of visual stimuli on the screen 10, i.e., the start time and the end time of the appearance of the visual stimuli.

In step S400, providing a feedback device 50, which comprises a plurality of push buttons 51 and a stop button 52, wherein the subject gives feedback on the perception of the displayed visual stimuli by pressing one of the push buttons 51. The subject can use the given push button 51 or several push buttons 51 to give feedback about the perceived start time and end time of the display of the visual stimulus and can also give feedback related to the perception of any other visual stimulus depending on the objectives of the test.

In step S500, providing a neuronal activity measuring device 40 with an internal clock 41, thereby registering the neuronal activity of said subject during the display of visual stimuli.

In step S600, providing a microcontroller 60, which comprises a real-time clock 61, thereby registering events of the optical sensor 30 and the feedback device 50 in real time, with millisecond precision, and generating a synchronizing time signal in connection with them.

In step S700, transmitting the events and neuronal activity registered by the internal clock 41 and the real-time clock 61, as well as the synchronizing time signals to the control unit 20.

Optionally, in a step S701, which is not shown in FIG. 3, the control unit 20 can transmit feedback to the screen 10. The feedback may be visually displayable feedback that provides clear feedback to the subject about the individual registered responses to the displayed visual stimuli. For example, the subject may provide feedback about the start of the presentation of a visual stimulus displayed on the screen 10 by pressing several push buttons 51 in a certain order. The control unit 20 is suitable for comparing the displayed visual stimulus and the given response, for providing feedback on the correctness of the response to the stimulus, and then for transmitting the feedback to the screen 10 in order to inform the subject. In this case, the control unit 20 does not yet synchronize the given stimulus and the associated response, but only provides intermediate feedback to the subject.

In step S800, using the synchronizing time signals to correlate at least the events registered by the real-time clock 61, preferably also the neuronal activity events registered by the internal clock 41.

In step S900, using the control unit 20 to combine the correlated events with each other, preferably with neuronal activities. The correlation and possible combination of individual events and neuronal activities with each other is described in detail in FIG. 4.

In step $1000, by the control unit 20, processing at least one, preferably all, of the following events and neuronal activities: the events and neuronal activities registered in steps S500 and S600, then transmitted to the control unit 20 in step S700, the events and neuronal activities correlated with each other in step S800 and/or combined in step S900 to determine the quantitative characteristics of the subject's psychophysical status. In FIG. 3, steps S700, S800 and S900 are demarcated by a dashed line to indicate the possibility of processing these steps in step S1000, that is, it is not automatically necessary to process all available data in step S1000. Quantitative characteristics determined during the processing can be, for example, the time elapsed between the display of the visual stimulus on the screen and the perception of the subject, the neuronal activity measured during the visual stimulation, preferably the peak time and the rise of the action potentials, etc. Depending on the experimental goals and the nature of the given test, different quantitative characteristics can be selected, which are part of the expected knowledge of the person skilled in the art, and therefore they are not described.

In step S1100, by the control unit 20, comparing the quantitative characteristics of the subject's psychophysical status with predetermined quantitative characteristics and/or the quantitative characteristics of psychophysical statuses stored in a database, wherein the database comprises quantitative characteristics of the psychophysical status of standard subjects and/or subjects suffering from a specific disease. The predetermined quantitative characteristics, as well as their value or range, can also be selected according to the given test. The collection of psychophysical statuses stored in the database and their quantitative characteristics can also be adjusted to the test, the database can comprise patient data related to one or more neurological diseases, or information that can be considered standard data related to a subject without a neurological disease.

In step S1200, based on the result of the comparison, clustering the quantitative characteristics to determine the subject's psychophysical status. As a result of clustering, i.e., assigning to a specific group, it is possible to determine the psychophysical status of the subject to be examined, optionally the minimum deviation of individual quantitative characteristics from the predetermined value or compared to the data stored in the database.

FIG. 4 shows a flow diagram of the steps of a method according to the invention. It should be noted that the steps shown in FIG. 4 can also be a part of the method shown in FIG. 3, namely steps S500-S800 of the method shown in FIG. 3, however, to avoid repetition, these steps are only described in detail in this section of the present description.

The method according to FIG. 4 can also be applied to a system 1 for synchronizing the biological data of a subject in real time, with millisecond precision. During the method according to FIG. 4, the real-time clock 61 of the microcontroller 60 can be used to register several events in real-time, with millisecond precision. In step S601a, using the real-time clock 61 of the microcontroller 60 to register the start times of the display of the visual stimuli measured by the optical sensor 30 and push button signals, which refer to the start times of the subject's visual perception, i.e., the time when the subject first perceives the visual stimulus displayed on the screen 10. In step S601b, generating a first synchronizing time signal for the registered start times of the display of each visual stimulus measured by the optical sensor 30, and a second synchronizing time signal for each registered push button signal, which are related to the start times of the subject's visual perception. In step S701, transmitting the registered start time events and the first and second synchronizing time signals to the control unit 20, and then in step S801, based on the first and second synchronizing time signals, correlating, i.e., real time matching the registered start time events with each other.

In the method according to FIG. 4, step S602a is performed in parallel with the previous ones, wherein using the real-time clock 61 of the microcontroller 60 to register end times of the display of the visual stimuli measured by the optical sensor 30 and push button signals for end times of visual perception of the subject, in real time, with millisecond precision. Subsequently, in step S602b, generating a third synchronization time signal for the registered end times of the display of each visual stimulus measured by the optical sensor 30 and a fourth synchronization time signal for the registered push button signals, which are related to the end times of the subject's visual perception. In step S702, transmitting the registered end time events and the third and fourth synchronization time signals to the control unit 20, and then in step S802, based on the third and fourth synchronization time signals, correlating the registered end time events.

In the method according to FIG. 4, the stop button 52 is also pressed, so in step S603a, using the real-time clock 61 of the microcontroller 60 to register, in real time, with millisecond precision, the time of pressing the stop button 52, and then in step S603b, generating a fifth synchronization time signal for registered time of pressing the stop button 52. After that, in step S703, transmitting the registered stop button event and the fifth synchronizing time signal to the control unit 20, and then in step S803, based on the third, fourth, and fifth synchronizing time signals, correlating the registered end time events and the registered stop button event, thus, a conclusion can be drawn about the subject's perception of the manually stopped visual stimulation.

In the method according to FIG. 4, in addition to the registration with the microcontroller 60, registration is also done with the neuronal activity measuring device 40. In step S501, using the internal clock 41 of the neuronal activity measuring device 40 to register the neuronal activity during each registered start time event, the neuronal activity during each registered end time event, and the action potentials measured during the display of each visual stimulus, especially their start times, end times and/or peak times. In step S704, transmitting the neuronal activity registered by the neuronal activity measuring device 40 to the control unit 20. Then, based on the first, second, third, fourth and/or fifth synchronizing time signal, in step S804, correlating the neuronal activity registered by the internal clock 41 of the neuronal activity measuring device 40 and at least one, preferably all, of the events registered by the real-time clock 61 of the microcontroller 60.

Correlating the above events and neuronal activities shows that events registered with the microcontroller 60 can be matched based on time, for example, the start of the display of a visual stimulus and the start of the subject's perception of the displayed visual stimulus can be determined in real time. The events registered in real time with the microcontroller 60 can be correlated with neuronal activity registered in non-real time with the neuronal activity measuring device 40, for example the start time events in the previous example can be paired with the neuronal activity measured at the start time of the appearance of the visual stimulus (time, peak of action potentials, etc.), and also with neuronal activity measured at the start time of the response to visual perception. From the point of view of the examination, it is particularly advantageous if the neuronal activity measured during the entire duration of the presentation of the visual stimuli is registered and, in addition to the correlation, the neuronal activity data is analyzed by a specialist to determine the psychophysical characteristics, status, and optionally even the diagnosis. Although a preferred method is shown in FIG. 4, wherein the start time events, end time events, stop button event and neuronal activities are all correlated, however, in the case of the method according to the invention, it is not automatically necessary to correlate all the above events, it is also possible to synchronize only start time events and neuronal activities.

According to FIG. 4, in step S900, combining the correlated events and neuronal activity. For example, the combining can be done as follows: the correlated start-time events and end-time events are combined, and in addition to the events registered with the 60 microcontroller, the neuronal activity is also combined, such as the correlated start-time events are combined with the neuronal activity measured at the start times, while the correlated end-time events are combined with neuronal activity measured at the end times. The most advantageous combination is when all registered data are taken into account to determine the psychophysical status. This can happen, on the one hand, by correlating all events with each other, and on the other hand, by combining all pairs of correlated events with each other, as well as with neuronal activity, preferably with neuronal activity measured during individual visual stimuli.

The steps S1000-S1200 according to FIG. 4 are the same as the steps S1000-S1200 according to FIG. 3, so they are not described in this figure in order to avoid repetition.

The advantage of the system and methods according to the invention is that it can register certain events, i.e., visual stimuli, responses to individual stimuli and neuronal activity measured during the stimulation, at the hardware level, in real time, with millisecond temporal precision.

The advantage of the solution according to the invention is that it is suitable for giving visual feedback to the subject about the correctness of the subject's responses to visual stimuli, which on the one hand provides continuous confirmation to the subject during the test, and on the other hand can provide additional information about the effect of a test with or without feedback on the performance and neuronal activity of the given subject. The use of feedback can help determine the subject's psychophysical status.

The advantage of the invention is that the subject can be diagnosed based on the specific psychophysical characteristics of the subject and/or psychophysical status determined by clustering, wherein preferably, in addition to establishing a specific disease, minimal differences in neurological characteristics can also be determined, which can contribute to the early detection of specific diseases.

Another advantage is that a personalized prevention and/or treatment plan can be created for the subject based on psychophysical statuses and/or diagnosis. The prevention plan essentially means the mitigation of the early signs of an incipient neurological disorder/disease, the prevention and/or improvement of said neurological disorder/disease, while the treatment plan means the medical treatment plan for a subject with a diagnosable disease.

The real-time event registration with millisecond precision enables the synchronization of individual events, i.e., their real-time pairing, and thus the determination of the quantitative characteristics of the subject's psychophysical status. With the system and method according to the invention, even minimal deviations in the psychophysical status can be detected, which would not be detectable with traditional stimulus and response measuring devices, as well as with neuronal signal recording devices due to the delay caused by the operation of the individual devices and data transmission.

1. A system (1) for synchronization of biological data in real time, with millisecond temporal precision, said system comprises:
a screen (10) for displaying visual stimuli;
a control unit (20) connected to the screen (10) for transmitting visual stimuli;
an optical sensor (30) connected to the screen (10) for detecting visual stimuli;
a neuronal activity measuring device (40) with an internal clock (41), which is connected to the control unit (20) for transmitting neuronal activity events registered by the internal clock (41);
feedback device (50), which comprises:
a plurality of push buttons (51) suitable for detecting responses of a subject to visual stimuli, and
a stop button (52) suitable for stopping the display of visual stimuli; wherein the system further comprises:
an optoisolator (70);
a microcontroller (60) with a real-time clock (61), the microcontroller (60)
being connected to the optical sensor (30) and the feedback device (50) for registering their events in real time, with millisecond precision, by the real-time clock (61), wherein the microcontroller (60) is configured to generate a synchronizing time signal for each registered event, and
being further connected to the control unit (20) for transmitting the registered events and the synchronizing time signals associated to said registered events, and
being further connected by a wire with the neural activity measuring device (40) through the optoisolator (70) arranged along said wire so as to interrupt galvanic connection between the microcontroller (60) and the neural activity measuring device (40);
wherein the control unit (20) is configured
to receive each event registered by the real-time clock (61) and each neuronal activity events registered by the internal clock (41), and to correlate said events based on the synchronizing time signals.

2. The system (1) according to claim 1, wherein the neuronal activity measuring device (40) is an EEG, EMG, intraoperative extracellular electrophysiological measuring device and/or any other device suitable for recording neuronal activity and having an internal clock (41) for temporal registration of neuronal activity.

3. The system (1) according to claim 1, wherein start times and end times of the display of the visual stimuli are detected by the optical sensor (30).

4. The system (1) according to claim 1, wherein the microcontroller (60) having the real-time clock (61) is configured to register at least the following events in real time, with millisecond precision, and produce the associated synchronizing time signal: the start times of the display of the visual stimuli measured by the optical sensor (30) and push button signals for the start times of visual perception of a subject.

5. The system (1) according to claim 1, wherein the microcontroller (60) having the real-time clock (61) is further configured to register the following events in real time, with millisecond precision, and produce the associated synchronizing time signal: the end times of the display of the visual stimuli measured by the optical sensor (30) and push button signals for the end times of visual perception of a subject, optionally time of the stop button signal.

6. The system (1) according to claim 1, wherein the neuronal activity measuring device (40) having the internal clock (41) is configured to register the following events: neuronal activity at the start times of the display of each visual stimulus, neuronal activity at the end times of the display of each visual stimulus, and action potentials measured during the display of each visual stimulus, especially start times, end times and/or peak times of the action potentials.

7. The system (1) according to claim 1, wherein the control unit (20) is connected to the microcontroller (60) via wired connection, preferably via a serial port.

8. The system (1) according to claim 7, wherein the connection between the microcontroller (60) and the neuronal activity measuring device (40) is a wired connection, preferably a trigger cable.

9. The system (1) according to claim 1, wherein the control unit (20) is configured to:
receive and correlate the registered events, the registered neuronal activity and the synchronizing time signals;
store, combine and process received and/or correlated events and neuronal activity.

10. A method to be performed in a system (1) for synchronization of biological data of a subject in real time, with millisecond temporal precision, said method comprising:
providing a control unit (20) for controlling visual stimuli (S100),
providing a screen (10) and transmitting the visual stimuli to the screen for being displayed (S200),
providing an optical sensor (30), the optical sensor (30) being used to detect the display of visual stimuli on the screen (10) (S300),
providing a feedback device (50), which comprises a plurality of push buttons (51) and a stop button (52), wherein by pressing one of the push buttons (51) (S400), the subject gives feedback on the perception of the displayed visual stimuli,
providing a neuronal activity measuring device (40) with an internal clock (41), thereby registering neuronal activity of said subject during the display of visual stimuli (S500),
providing a microcontroller (60), which comprises a real-time clock (61), thereby registering events of the optical sensor (30) and the feedback device (50) in real time, with millisecond precision, and generating a synchronizing time signal for each of said events (S600),
transmitting the events and the neuronal activity registered by the internal clock (41) and the real-time clock (61), respectively, as well as the synchronizing time signals to the control unit (20) (S700),
using the synchronizing time signals to correlate at least the events registered by the real-time clock (61), and preferably also events of neuronal activity registered by the internal clock (41) (S800).

11. The method according to claim 10, comprising:
using the real-time clock (61) of the microcontroller (60) to register, in real time, with millisecond precision, start times of the display of the visual stimuli measured by the optical sensor (30) and push button signals for start times of visual perception of the subject (S601a),
generating a first synchronizing time signal for the registered start times of the display of each visual stimulus measured by the optical sensor (30), and a second synchronizing time signal for each registered push button signal, which are related to the start times of the subject's visual perception (S601b),
transmitting the registered start time events and the first and second synchronizing time signals to the control unit (20) (S701),
based on the first and second synchronizing time signals, correlating the registered start time events by the control unit (20) (S801).

12. The method according to claim 10, further comprising:
using the real-time clock (61) of the microcontroller (60) to register, in real time, with millisecond precision, end times of the display of visual stimuli measured by the optical sensor (30) and push button signals for end times of visual perception of the subject (S602a),
generating a third synchronizing time signal for the registered end times of the display of each visual stimulus measured by the optical sensor (30), and a fourth synchronizing time signal for the registered push button signals, which are related to the end times of the subject's visual perception (S602b),
transmitting the registered end time events and the third and fourth synchronization time signals to the control unit (20) (S702),
based on the third and fourth synchronizing time signals, correlating the registered end time events by the control unit (20) (S802).

13. The method according to claim 12, further comprising:
using the real-time clock (61) of the microcontroller (60) to register, in real time, with millisecond precision, time of pressing the stop button (52) (S603a),
generating a fifth synchronizing time signal for registered time of pressing the stop button (52) (S603b),
transmitting the registered stop button event and the fifth synchronization time signal to the control unit (20) (S703),
based on the third, fourth and fifth synchronizing time signals, correlating the registered end time events and the registered stop button event by the control unit (20) (S803).

14. The method according to claim 13, further comprising:
using the internal clock (41) of the neuronal activity measuring device (40) to register the neuronal activity during each registered start time event, the neuronal activity during each registered end time events, and particularly start times, end times and/or peak times of the action potentials measured during the display of each visual stimulus (S501),
transmitting the neuronal activity registered by the neuronal activity measuring device

(40) to the control unit (20) (S704),
based on the first, second, third, fourth and/or fifth synchronizing time signal, by the control unit (20), correlating the neuronal activity registered by the internal clock (41) of the neuronal activity measuring device (40) and at least one of the events registered by the real-time clock (61) of the microcontroller (60) (S804).

15. The method according to claim 14, comprising:
by the control unit (20), combining the correlated events and neuronal activity, preferably combining the events and neuronal activity that are correlated based on the first, second, third, fourth and fifth synchronizing time signals (S900).

16. (canceled)

17. (canceled)

18. A method to be performed by the system (1) according to claim 1, for synchronizing of biological data of a subject in real time, with millisecond precision, said method comprising:
using real-time clock (61) of the microcontroller (60) to register in real time, with millisecond precision, start times of the display of the visual stimuli measured by the optical sensor (30) and push button signals for start times of the visual perception of the subject (S601a),
generating a first synchronizing time signal for each registered start times of the display of each visual stimulus measured by the optical sensor (30), and a second synchronizing time signal for each registered push button signal being related to the start time of the subject's visual perception (S601b),
transmitting the registered start time events and the first and second synchronizing time signals to the control unit (20) (S701),
based on the first and second synchronizing time signals, correlating the registered start time events by the control unit (20) (S801).

19. The method according to claim 18, further comprising:
using the real-time clock (61) of the microcontroller (60) to register, in real time, with millisecond precision, end times of the display of visual stimuli measured by the optical sensor (30) and push button signals for end times of the visual perception of the subject (S602a),
generating a third synchronizing time signal for the registered end times of the display of each visual stimulus measured by the optical sensor (30), and a fourth synchronizing time signal for the registered push button signals, which are related to the end times of the subject's visual perception (S602b),
transmitting the registered end time events and the third and fourth synchronization time signals to the control unit (20) (S702),
based on the third and fourth synchronizing time signals, correlating the registered end time events by the control unit (20) (S802), and optionally,
using the real-time clock (61) of the microcontroller (60) to register, in real time, with millisecond precision, time of pressing the stop button (52) (S603a),
generating a fifth synchronizing time signal for registered time of pressing the stop button (52) (S603b),
transmitting the registered stop button event and the fifth synchronization time signal to the control unit (20) (S703), and
based on the third, fourth, and optionally fifth synchronizing time signals, correlating the registered end time events and the registered stop button event by the control unit (20) (S803).

20. (canceled)

21. The method according to claim 18, further comprising:
using the internal clock (41) of the neuronal activity measuring device (40) to register the neuronal activity related to each registered start time event of the action potentials measured during the display of each visual stimulus (S501),
transmitting the neuronal activity registered by the neuronal activity measuring device (40) to the control unit (20) (S704),
by the control unit (20), based on the first and second synchronizing time signal, correlating the neuronal activity registered by the internal clock (41) of the neuronal activity measuring device (40) and at least one of the events registered by the real-time clock (61) of the microcontroller (60) (S804).

22. (canceled)

23. (canceled)

24. (canceled)

25. The method according to claim 19, further comprising:
using the internal clock (41) of the neuronal activity measuring device (40) to register the neuronal activity related to each registered start time event, and the neuronal activity related to each registered end time event, in particular the start times, end times and/or peak times of the action potentials measured during the display of each visual stimulus (S501),
transmitting the neuronal activity registered by the neuronal activity measuring device (40) to the control unit (20) (S704),
by the control unit (20), based on the first, second, third, fourth and optionally fifth synchronizing time signal, correlating the neuronal activity registered by the internal clock (41) of the neuronal activity measuring device (40) and the related events registered by the real-time clock (61) of the microcontroller (60) (S804),
and preferably, by the control unit (20), combining the events and neuronal activity that are correlated based on the first, second, third, fourth and optionally fifth synchronizing time signals (S900).

26. The method according to claim 21, further comprising:
using the internal clock (41) of the neuronal activity measuring device (40) to register the neuronal activity related to each registered start time event, and the neuronal activity related to each registered end time event, in particular the start times, end times and/or peak times of the action potentials measured during the display of each visual stimulus (S501),
transmitting the neuronal activity registered by the neuronal activity measuring device (40) to the control unit (20) (S704),
by the control unit (20), based on the first, second, third, fourth and optionally fifth synchronizing time signal, correlating the neuronal activity registered by the internal clock (41) of the neuronal activity measuring device (40) and the related events registered by the real-time clock (61) of the microcontroller (60) (S804),
and preferably, by the control unit (20), combining the events and neuronal activity that are correlated based on the first, second, third, fourth and optionally fifth synchronizing time signals (S900).

27. The method according to claim 21, further comprising:
processing at least one, preferably all, of the registered, correlated and/or combined events and neuronal activity to determine quantitative characteristics of the subject's psychophysical status (S1000), and/or
comparing the quantitative characteristics of the subject's psychophysical status with predetermined quantitative characteristics and/or quantitative characteristics of psychophysical statuses stored in a database, wherein the database comprises quantitative characteristics of the psychophysical status of standard subjects and/or subjects suffering from a given disease (S1100), and
based on the result of the comparison, clustering the quantitative characteristics in order to determine the subject's psychophysical status (S1200).
